(19) United States
(12) Patent Application Publication
(10) Pub. No.: US 2022/0002373 A1
(43) Pub. Date: Jan. 6, 2022

(54) SINGLE-CHAIN INSULIN ANALOGUES WITH POLY-ALANINE C-DOMAIN SUB-SEGMENTS

(71) Applicant: CASE WESTERN RESERVE UNIVERSITY, Cleveland, OH (US)

(72) Inventor: Michael A. WEISS, Indianapolis, IN (US)

(21) Appl. No.: 17/294,948

(22) PCT Filed: Nov. 19, 2019
(86) PCT No.: PCT/US2019/062259

§ 371 Date: May 18, 2021

Related U.S. Application Data
(60) Provisional application No. 62/769,324, filed on Nov. 19, 2018.

(51) Int. Cl. C07K 14/62

(52) U.S. Cl. CPC C07K 14/62; A61K 38/00

(57) ABSTRACT

A single-chain insulin analogue containing an engineered C-domain segment of lengths 4-11 conforming to the sequence pattern [Asp/Glu]-Ala-Aₙ-Ala-Xaa where Aₙ designates a sub-segment of 0-7 Alanine residues and where Xaa designates an amino-acid residue selected from the amino acids Alanine, Arginine, Asparagine, Aspartic Acid, Glutamic Acid, Histine, Lysine and Serine. The analogue may be an analogue of a mammalian insulin, such as human insulin, may optionally include standard or non-standard modifications that (i) augment the stability of insulin, (ii) cause a shift in the isoelectric point to enhance or impair the solubilty of the protein at neutral pH or (iii) reduce cross-binding of the protein to the Type I IGF receptor. A method of treating a patient with diabetes mellitus comprising the administration of a physiologically effective amount of the protein or a physiologically acceptable salt thereof to a patient.

### CROSS REFERENCE TO RELATED APPLICATION

This application is a national stage application of PCT/US2019/062259, filed on Nov. 19, 2019, which claims benefit of U.S. Provisional Application No. 62/769,324, filed on Nov. 19, 2018. The disclosures of the above-referenced applications are incorporated herein by reference.

### STATEMENT REGARDING FEDERALLY SPONSORED RESEARCH OR DEVELOPMENT

This invention was made with government support under grant numbers DK040949 and DK074176 awarded by the National Institutes of Health. The government has certain rights to the invention.

### BACKGROUND OF THE INVENTION

This invention relates to polypeptide hormone analogues that exhibit enhanced pharmaceutical properties, such as increased increased thermodynamic stability, augmented resistance to thermal fibrillation above room temperature, decreased mitogenicity, and/or altered pharmacokinetic and pharmacodynamic properties, i.e., conferring more prolonged duration of action or more rapid duration of action relative to soluble formulations of the corresponding wild-type human hormone. More particularly, this invention relates to insulin analogues consisting of a single polypeptide chain that contains a novel class of foreshortened connecting (C) domains between A and B domains. Of length 4-11 residues, the C domains of this class consist of an N-terminal acidic element and a C-terminal segment containing at least one basic amino-acid residue. The single-chain insulin analogues of the present invention may optionally contain standard or non-standard amino-acid substitutions at other sites in the A or B domains.

The engineering of non-standard proteins, including therapeutic agents and vaccines, may have broad medical and societal benefits. Naturally occurring proteins—as encoded in the genomes of human beings, other mammals, vertebrate organisms, invertebrate organisms, or eukaryotic cells in general—often confer multiple biological activities. A benefit of non-standard proteins would be to achieve augmented resistance to degradation at or above room temperature, facilitating transport, distribution, and use. An example of a therapeutic protein is provided by insulin. Wild-type human insulin and insulin molecules encoded in the genomes of other mammals bind to insulin receptors in multiple organs and diverse types of cells, irrespective of the receptor isoform generated by alternative modes of RNA splicing or by alternative patterns of post-translational glycosylation. Wild-type insulin also binds with lower affinity to the homologous Type 1 insulin-like growth factor receptor (IGF-1R).

An example of a further medical benefit would be optimization of the stability of a protein toward unfolding or degradation. Such a societal benefit would be enhanced by the engineering of proteins that are more refractory than standard proteins with respect to degradation at or above room temperature, particularly for use in regions of the developing world where electricity and refrigeration are not consistently available. Analogues of insulin consisting of a single polypeptide chain and optionally containing non-standard amino-acid substitutions may exhibit superior properties with respect to resistance to thermal degradation or decreased mitogenicity. The challenge posed by its physical degradation is deepened by the developing epidemic of diabetes mellitus in Africa and Asia. Because fibrillation poses the major route of degradation above room temperature, the design of fibrillation-resistant formulations may enhance the safety and efficacy of insulin replacement therapy in such challenged regions.

Administration of insulin has long been established as a treatment for diabetes mellitus. A major goal of conventional insulin replacement therapy in patients with diabetes mellitus is tight control of the blood glucose concentration to prevent its excursion above or below the normal range characteristic of healthy human subjects. Excursions below the normal range are associated with immediate adrenergic or neuroglycopenic symptoms, which in severe episodes lead to convulsions, coma, and death. Excursions above the normal range are associated with increased long-term risk of microvascular disease, including retinapathy, blindness, and renal failure.

Insulin is a small globular protein that plays a central role in metabolism in vertebrates. Insulin contains two chains, an A chain, containing 21 residues, and a B chain containing 30 residues. The hormone is stored in the pancreatic β-cell as a Zn²⁺-stabilized hexamer, but functions as a Zn²⁺-free monomer in the bloodstream. Insulin is the product of a single-chain precursor, proinsulin, in which a connecting region (35 residues) links the C-terminal residue of B chain (residue B30) to the N-terminal residue of the A chain (FIG. 1A). A variety of evidence indicates that it consists of an insulin-like core and disordered connecting peptide (FIG. 1B). Formation of three specific disulfide bridges (A6-A11, A7-B7, and A20-B19; FIGS. 1A and 1B) is thought to be coupled to oxidative folding of proinsulin in the rough endoplasmic reticulum (ER). Proinsulin assembles to form soluble Zn′-coordinated hexamers shortly after export from ER to the Golgi apparatus. Endoproteolytic digestion and conversion to insulin occurs in immature secretory granules followed by morphological condensation. Crystalline arrays of zinc insulin hexamers within mature storage granules have been visualized by electron microscopy (EM). The sequence of insulin is shown in schematic form in FIG. 1C. Individual residues are indicated by the identity of the amino acid (typically using a standard one-letter or three-letter code), the chain and sequence position (typically as a superscript). Pertinent to the present invention is the invention of novel foreshortened C domains of length 4-11 residues in place of the 36-residue wild-type C domain characteristic of human proinsulin.

Fibrillation, which is a serious concern in the manufacture, storage and use of insulin and insulin analogues for the treatment of diabetes mellitus, is enhanced with higher temperature, lower pH, agitation, or the presence of urea, guanidine, ethanol co-solvent, or hydrophobic surfaces. Current US drug regulations demand that insulin be discarded if fibrillation occurs at a level of one percent or more. Because fibrillation is enhanced at higher temperatures, patients with diabetes mellitus optimally must keep insulin refrigerated prior to use. Fibrillation of insulin or an insulin analogue can be a particular concern for such patients utilizing an external insulin pump, in which small amounts of insulin or insulin analogue are injected into the patient's body at regular intervals. In such a usage, the insulin or insulin analogue is not kept refrigerated within the pump apparatus, and fibrillation of insulin can result in blockage of the catheter used to inject insulin or insulin analogue into the body, potentially resulting in unpredictable fluctuations in blood glucose levels or even dangerous hyperglycemia. Insulin exhibits an increase in degradation rate of 10-fold or more for each 10° C. increment in temperature above 25° C.; accordingly, guidelines call for storage at temperatures <30° C. and preferably with refrigeration. Fibrillation of basal insulin analogues formulated as soluble solutions at pH less than 5 (such as Lantus® (Sanofi-Aventis), which contains an unbuffered solution of insulin glargine and zinc ions at pH 4.0) also can limit their shelf lives due to physical degradation at or above room temperature; the acidic conditions employed in such formulations impairs insulin self-assembly and weakens the binding of zinc ions, reducing the extent to which the insulin analogues can be protected by sequestration within zinc-protein assemblies.

Insulin is susceptible to chemical degradation, involving the breakage of chemical bonds with loss of rearrangement of atoms within the molecule or the formation of chemical bonds between different insulin molecules. Such changes in chemical bonds are ordinarily mediated in the unfolded state of the protein, and so modifications of insulin that augment its thermodynamic stability also are likely to delay or prevent chemical degradation.

Insulin is also susceptible to physical degradation. The present theory of protein fibrillation posits that the mechanism of fibrillation proceeds via a partially folded intermediate state, which in turn aggregates to form an amyloidogenic nucleus. According to this theory, it is possible that amino-acid substitutions that stabilize the native state may or may not stabilize the partially folded intermediate state and may or may not increase (or decrease) the free-energy barrier between the native state and the intermediate state. Therefore, the current theory indicates that the tendency of a given amino-acid substitution in the two-chain insulin molecule to increase or decrease the risk of fibrillation is highly unpredictable. Models of the structure of the insulin molecule envisage near-complete unfolding of the three-alpha helices (as seen in the native state) with parallel arrangements of beta-sheets formed successive stacking of B-chains and successive stacking of A-chains; native disulfide pairing between chains and within the A-chain is retained. Such parallel cross-beta sheets require substantial separation between the N-terminus of the A-chain and C-terminus of the B-chain (>30 Å), termini ordinarily in close proximity in the native state of the insulin monomer (<10 Å).

Marked resistance to fibrillation of single-chain insulin analogues with foreshortened C-domains is known in the art and thought to reflect a topological incompatibility between the splayed structure of parallel cross-beta sheets in an insulin protofilament and the structure of a single-chain insulin analogue with native disulfide pairing in which the foreshortened C-domain constrains the distance between the N-terminus of the A-chain and C-terminus of the B-chain to be unfavorable in a protofilament. The three-dimensional structure of an active and ultra-stable single-chain insulin, 57 residues in length and containing C-domain Gly-Gly-Gly-Pro-Arg-Arg (GGGPRR; SEQ ID NO: 7), is shown in FIG. 2 in relation to an inactive single-chain analogue that contains a peptide bind between Lys^{B29} and Gly^{A1} (i.e., des-B30 SCI of 50 residues in length).

The present invention addresses the need to optimize the augmented stability conferred upon insulin by chemical tethers between the A and B chains (e.g., between the c-amino group of Lys^{B29} and the a-amino group of Gly^{A1}) and by foreshortened C domains. The latter analogues are designated single-chain insulin analogues (SCIs). Whereas direct peptide bonds from residues at or near the C-terminal end of the B chain (residues B28, B29 or B30) to Gly generally result in marked impairment of biological activity, foreshortened C domains of length 4-11 provide sufficient conformational “play” to permit at least a substantial portion of receptor-binding affinity. The structural basis of such length discrimination has been suggested by the crystal structure of a “micro-receptor”/insulin complex containing a ternary complex between the hormone and two portions of the ectodomain of the insulin receptor: the N-terminal fragment L1-CR and the C-terminal fragment αCT. SCIs can in general exhibit prolonged signaling once in the bloodstream, an unfavorable pharmacodynamic property in relation to use in insulin pumps and mealtime insulin replacement therapy. Ultra-stable single-chain or two-chain insulin analogues known in the art have exhibited a puzzling aberrant prolongation of signaling as tested on intravenous bolus injection in diabetic rats (FIG. 3). To our knowledge, no rules are known that can predict the pharmacodynamic effects of mixed-sequence C-domains (such as Glu-Glu-Gly-Pro-Arg-Arg [EEGPRR]), which can confer biphasic pharmacodynamics properties in one B-domain context but not another.

There is a need, therefore, for single-chain insulin analogues containing simplified C-domain sequences such that their biological and biophysical properties would be readily optimized for favorable therapeutic applications. Examples of such applications are provided by (i) rapid action in performance in external or internal insulin pumps and (ii) biphasic action in ultra-stable and mono-component soluble formulations for use in challenged regions of the developing world. There would be a particular need for simplified C-domain sequences in which the SCI's rapid action on subcutaneous injection in a diabetic mammal would be maintained in the presence of stabilizing substitutions in the A or B domains and/or in the presence of B-domain substitutions known in the art to accelerate the absorption of two-chain insulin analogs.

### SUMMARY OF THE INVENTION

It is, therefore, an aspect of the present invention to provide single-chain insulin analogues that contain simplified C-domain sequences with internal poly-Alanine sub-segments. These sequences are of length 4-11 and contain an N-terminal acidic residue (Aspartic Acid or Glutamic Acid) and a C-terminal residue that is either Alanine, an acidic residue or basic residue (Group X: Alanine, Arginine, Asparagine, Aspartic Acid, Glutamic Acid, Histine, Lysine or Serine). These C-domain sequences thus conform to the pattern [Asp/Glu]-Ala-Aₙ-Ala-[Xaa] where Aₙ designates a sub-segment of 0-7 Alanine residues and where Xaa designates an amino-acid residue selected from Group X above (SEQ ID NO: 9). It is an additional aspect of the present invention that absolute in vitro affinities of the single-chain insulin analogue for IR-A and IR-B are in the range 5-150% relative to wild-type human insulin and so unlikely to exhibit significantly prolonged residence times in the hormone-receptor complex. It is yet another aspect of the present invention that such optimized analogues should bind more weakly to the mitogenic IGF-1R receptor than does wild-type human insulin, which would tend to indicate reduced mitogenicity in mammalian cell culture. The present invention addresses the utility of single-chain insulin analogues whose simplified C-domain sequences facilitate co-optimization of biophysical, biological and pharmacodynamic features that are favorable for therapeutic applications.

### BRIEF DESCRIPTION OF THE SEVERAL VIEWS OF THE DRAWINGS

FIG. 1A is a schematic representation of the sequence of human proinsulin including the A- and B-chains and the connecting region shown with flanking dibasic cleavage sites (filled circles) and C-peptide (open circles).

FIG. 1B is a structural model of proinsulin, consisting of an insulin-like moiety and a disordered connecting peptide (dashed line).

FIG. 1C is a schematic representation of the sequence of human insulin indicating the position of residues B27 and B30 in the B-chain.

FIG. 2 depicts the solution structure of SCI-c (PDB: 2JZQ (29)) with A domain, B domain, C domain, and native disulfides shown.

FIG. 3A is a ribbon structure representation of [Asp^{B10}, Lys^{B28}, Pro^{B29}, Cys^{B4}, Cys^{A10}]-insulin (“4SS-DKP”; SEQ ID NOS: 10 and 11) with the asterisk denoting a fourth disulfide bridge.

FIG. 3B is a ribbon structure representation of SCI-c of Hua, Q. X., et al. ((2008) “Design of an active ultrastable single-chain insulin analog: synthesis, structure, and therapeutic implications.” J. Biol. Chem. 283: 14703-14716).

FIG. 3C is a graphic representation of insulin action over time for 4SS-DKP-insulin relative to WT insulin and a diluent according to Vinther et al. ((2013) “Insulin analog with additional disulfide bond has increased stability and preserved activity.” Protein Sci. 22:296-305) in diabetic rats.

FIG. 3D is a graphic representation of insulin action obtained after IV injection of insulin lispro (SEQ ID NOS: 2 and 12; N=13), 4SS-DKP-insulin (SEQ ID NOS: 10 and 11; N=10), [Asp^{B10}, Lys^{B28}, Pro^{B29}]-insulin (“DKP”; SEQ ID NO: 13; N=9), and SCI-c (N=8). A negative control was provided by IV injection of Lilly Diluent (violet; N=12). Insulin doses were each 1.7 nmol/300 g rat.

FIG. 4 provides a schematic illustration of the simplified C domains of the present invention Containing and N-terminal element (E-A-), poly-Alanine subsegments (Aₙ) containing n=0, 1, 2, 3, 4, 5, 6 or 7 residues, and a C-terminal element, A-X, (where X is selected from a subset of amino acids consisting of Alanine, Arginine, Asparagine, Aspartic Acid, Glutamic Acid, Histine, Lysine or Serine; SEQ ID NO: 9).

FIG. 5 provides a schematic illustration of the A1-A8 α-helix in native insulin (shown as a cylinder with sequence underneath; residues 1-8 of SEQ ID NO: 2) highlighting electrostatic features pertinent to the present invention. The wild-type sequence contains a central acidic residue (Glu^{A4}) whose negative charge may participate in (i, i−4) and (i, i+4) electrostatic interactions; the former would involve the C-terminal side chain of the C domain whereas the latter could involve a variant side chain at position A8. Examples of A4-related electrostatic interactions would be provided by (a) an Arg, His or Lys at the C-terminal position of the C domain or (b) an Arg, His or Lys at position A8. Asn, Asp, Glu or Ser at the C-terminal position of the C domain may provide a favorable N-Cap of this helix whereas Arg, His or Lys at position A8 may provide a C-Cap more favorable than the wild-type Thr^{A8}.

FIG. 6 provides the results of biological testing in Sprague-Dawley rats made diabetic with streptozotocin: subcutaneous injection of three SCIs were studied in relation to a control subcutaneous injection of insulin lispro (SEQ ID NOS: 2 and 12; N=6 per group). The dose was 15 μg of insulin lispro per 300-gram rat; doses of the SCIs were equivalent in units of nanomoles per 300-gram rat (i.e., micrograms corrected for respective molecular masses). Results depict mean blood-glucose concentration (vertical axis) as a function of time in minutes (horizontal axis). Symbol code (box at upper right): (filled square, [symbol] ) fresh insulin lispro as positive control; (filled triangle, [symbol] ) fresh SCI-2 (SEQ ID NO: 5; linker EEGPRR with A-domain substitutions Thr^{A8} [symbol] His and Tyr^{A14} [symbol] Glu and with B-domain substitutions Pro^{B28} [symbol] Asp and Lys^{B29} [symbol] Pro); (filled circle, ●) SCI-3 (SEQ ID NO: 6; variant of SCI-2 in which His^{A8} was reverted to wild-type Thr and Glu^{A14} was reverted to wild-type Tyr); and (filled diamond, ♦) SCI-4 (SEQ ID NO: 4) with linker EAAAAA in the context of His^{A8}, Tyr^{A14}, Asp^{B28} and Pro^{B29}.

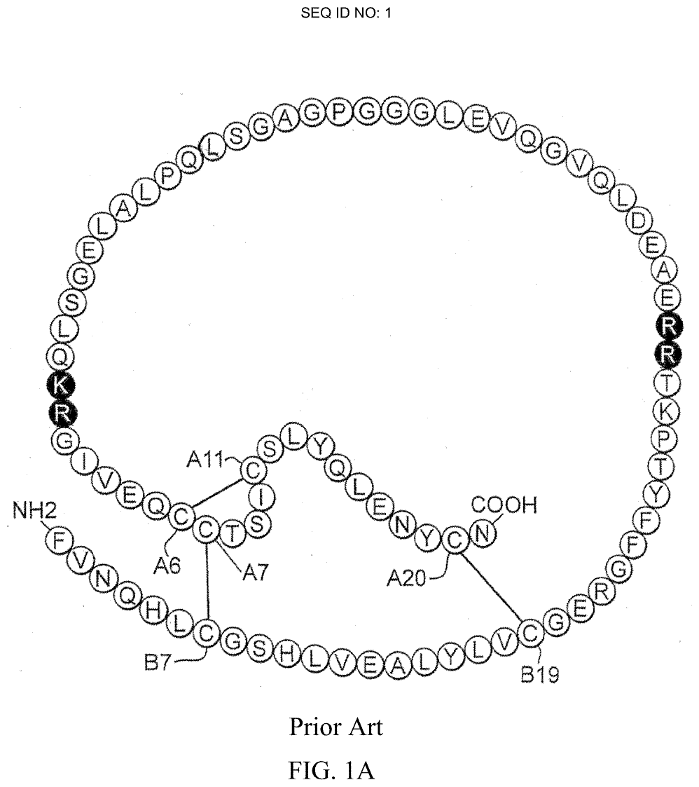

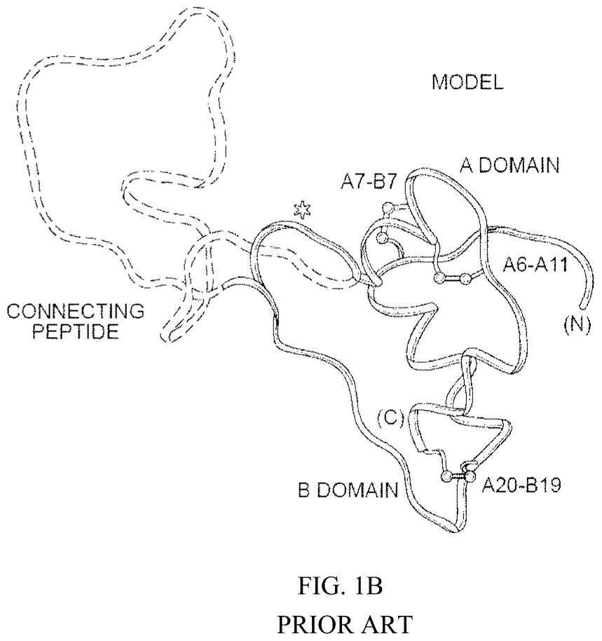

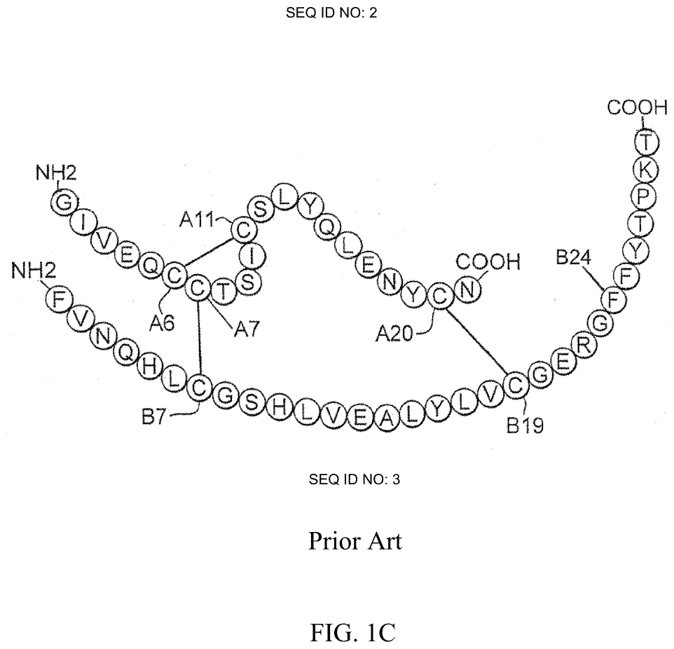

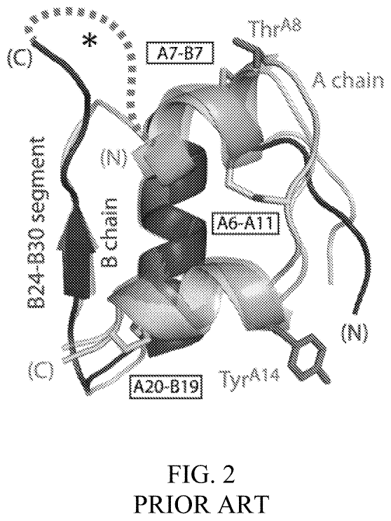

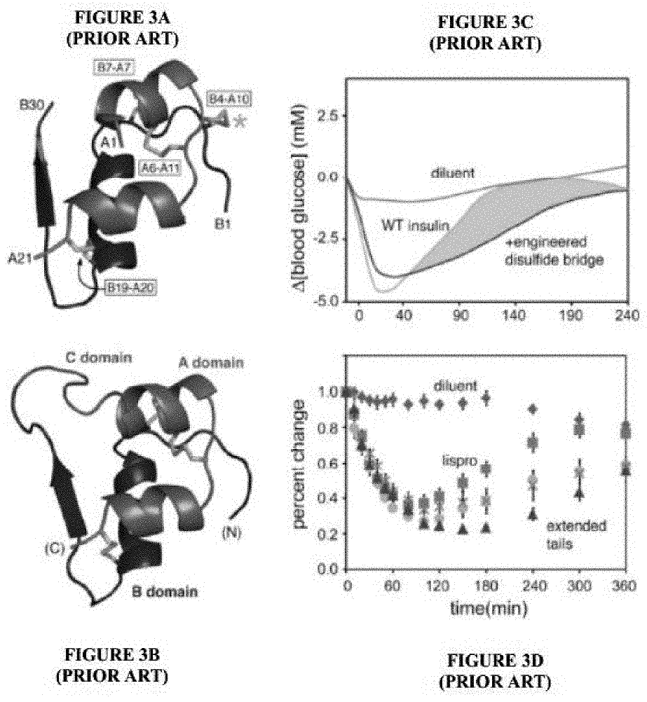

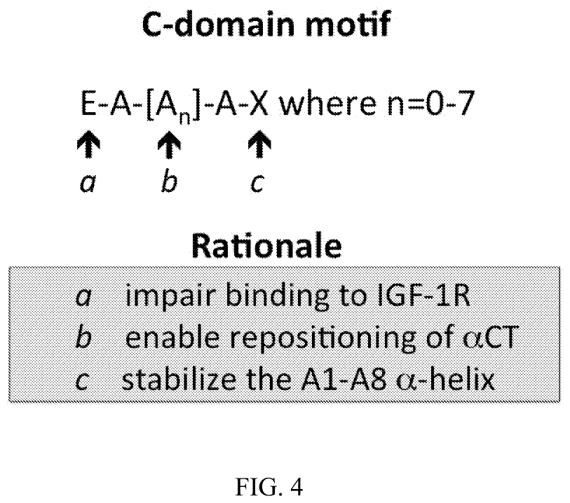

### DETAILED DESCRIPTION OF THE INVENTION

The present invention is directed toward a single-chain insulin analogue that provides (i) enhanced stability and resistance to fibrillation due to the presence of a simplified and foreshortened C domain (length 4-11 residues) and (ii) ready and convenient co-optimization of biological, biophysical and pharmacodynamics properties. The single-chain insulin analogues of the present invention may have an isoelectric point between 4.0 and 6.0 (and so be suitable for formulation under neutral pH conditions as a rapid-acting insulin analogue formulation) or may have an isoelectric point between 6.5 and 8.0 (and so be suitable for formulation under acidic pH conditions as a basal insulin analogue formulation). A molecular embodiment of this strategy were prepared by biosynthetic expression in the yeast Pichia pastoris, designated SCI-4, whose properties stand in relation to SCIs known in the art (designated SCI-1, SCI-2 and SCI-3).

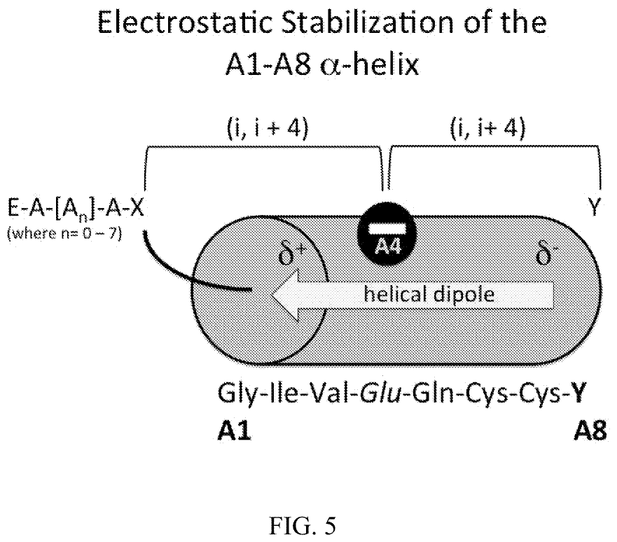

The foreshortened C domains of the present invention are shown in schematic form in FIG. 5. The A1-A8 α-helix in native insulin (shown as a cylinder with sequence underneath) highlights electrostatic features pertinent to the present invention. The wild-type sequence contains a central acidic residue (Glu^{A4}) whose negative charge may participate in (i, i−4) and (i, i+4) electrostatic interactions. The former would involve the C-terminal side chain of the C domain whereas the latter could involve a variant side chain at position A8. Examples of A4-related electrostatic interactions would be provided by (a) an Arg, His or Lys at the C-terminal position of the C domain or (b) an Arg, His or Lys at position A8. Asn, Asp, Glu or Ser at the C-terminal position of the C domain may provide a favorable N-Cap of this helix whereas Arg, His or Lys at position A8 may provide a C-Cap more favorable than the wild-type Thr^{A8}. The N-terminal residue of the C domain is acidic (Aspartic Acid or Glutamic Acid) in order to impair binding of the analogues to the mitogenic Type 1 IGF receptor (IGF-1R) relative to insulin receptor isoforms (IR-A and IR-B).

The C-terminal residue of the C domain provides a “tuneable knob” to facilitate co-optimization of an analogue's isoelectric point (and hence pH-dependent solubility), thermodynamic stability, segmental A1-A8 helical dynamics, pharmacokinetic properties and pharmacodynamics properties. While not intending to be constrained by theory, this knob is positioned to interact through electrostatic interactions with the helical dipole axis (horizontal arrow in FIG. 5), including as a potential N-Cap residue, and with the negative charge of Glu′ via (i, i−4) side-chain interactions. Further modulation of the electrostatic features of this a-helical segment can be provided by substitutions at position A8 via (i, i+4) side-chain interactions. The wild-type residue at position A8, which is Threonine in human insulin, contains a β-branched side chain that is suboptimal with respect to both a-helical propensity and C-Cap propensity. The smallest such C domain contains four residues with central di-Alanine element ([Asp/Glu-Ala-Ala-Xaa]) whereas the longest such C domain contains 11 residues with a 9-residue poly-Alanine sub-segment.

The single-chain insulin analogues of the present invention may also contain substitutions within their respective A- and B domains. B-domain substitutions can include variants known in the art to weaken self-association and thus confer rapid absorption on subcutaneous injection; examples include AspB28 (as in Novolog®; insulin aspart), Lys^{B28}-Pro^{B29} (as in Humalog®; insulin lispro) or Asp^{B28}-Pro^{B29}. Substitution of hyper-exposed Tyr^{A14} by Glu may mitigate an unfavorable “reverse-hydrophobic effect”—thereby augmenting thermodynamic stability—and simultaneously remove a potential aromatic site of chemical degradation. The analogues of the present invention exclude the substitution His^{B10}→Asp, which has been associated with enhanced mitogenicity in cell culture and carcinogenesis in rat testing.

In view of the similarity between human and animal insulins, and use in the past of animal insulins in human patients with diabetes mellitus, it is also envisioned that other minor modifications in the sequence of insulin may be introduced, especially those substitutions considered “conservative.” For example, additional substitutions of amino acids may be made within groups of amino acids with similar side chains, without departing from the present invention. These include the neutral hydrophobic amino acids: Alanine (Ala or A), Valine (Val or V), Leucine (Leu or L), Isoleucine (Ile or I), Proline (Pro or P), Tryptophan (Trp or W), Phenylalanine (Phe or F) and Methionine (Met or M). Likewise, the neutral polar amino acids may be substituted for each other within their group of Glycine (Gly or G), Serine (Ser or S), Threonine (Thr or T), Tyrosine (Tyr or Y), Cysteine (Cys or C), Glutamine (Glu or Q), and Asparagine (Asn or N). Basic amino acids are considered to include Lysine (Lys or K), Arginine (Arg or R) and Histidine (His or H). Acidic amino acids are Aspartic acid (Asp or D) and Glutamic acid (Glu or E). Unless noted otherwise or wherever obvious from the context, the amino acids noted herein should be considered to be L-amino acids. Standard amino acids may also be substituted by non-standard amino acids belong to the same chemical class. By way of non-limiting example, the basic side chain Lys may be replaced by basic amino acids of shorter side-chain length (Ornithine, Di-aminobutyric acid, or Di-aminopropionic acid). Lys may also be replaced by the neutral aliphatic isostere Norleucine (Nle), which may in turn be substituted by analogues containing shorter aliphatic side chains (Aminobutyric acid or Aminopropionic acid).

A representative analogue of the present invention was purified from an engineered strain of the yeast Pichia pastoris as described (Glidden, M. D., et al. 2017ab, “Solution structure of an ultra-stable single-chain insulin analog connects protein dynamics to a novel mechanism of receptor binding.” J. Biol. Chem. pii: jbc.M117.808667. doi: 10.1074/jbc.M117.808667 [Epub ahead of print]). This analogue, 57 residues in length, contains a C domain of sequence Glu-Ala-Ala-Ala-Ala-Ala (EAAAAA) and thus conforms to the template Glu-Ala-Aₙ-Xaa where n=2 (underlined above) and where Xaa is also Alanine (SEQ ID NO: 13). The analogue (designated SCI-4; SEQ ID NO: 4) contained four substitutions in the insulin moiety: Thr^{A8} [symbol] His and Tyr^{A14} [symbol] Glu and with B-domain substitutions Pro^{B28} [symbol] Asp and Lys^{B29} [symbol] Pro. The respective rationales for these substitutions were as follows. His^{A8} was introduced to augment receptor-binding affinity and thermodynamic stability; Glu^{A14} was introduced to enhance stability and reduce the isoelectric point (otherwise increased by the partial charge of His^{A8}); and the Asp^{B28}-Pro^{B29} element was introduced to weaken dimerization and further reduce the isoelectric point.

The connecting domain or C-domain of SCI-4 has a general structure comprising an N-terminal [Asp/Glu]-Ala element which functions to impair binding of the SCI to the mitogenic Type 1 IGF receptor (IGF-1R). The C-terminal element Ala-Xaa (where Xaa is selected from a subset of amino acids consisting of Alanine, Arginine, Asparagine, Aspartic Acid, Glutamic Acid, Histine, Lysine or Serine) provides a tuneable knob to modulate the analog's isoelectric point, stability, segmental helical stability, and pharmacodynamic properties. The foreshortened C domains of the present invention optionally contain poly-Alanine subsegments (Aₙ) containing n=0, 1, 2, 3, 4, 5, 6 or 7 residues. The total length of the C domain is thus in the range 4-11 residues. The shortest C domain contains a central di-Alanine subsegment ([Asp/Glu]-Ala-Ala) whereas the longest C domain contains a central poly-Alanine subsegment containing 7 alanines to yield 9 total Alanine residues.

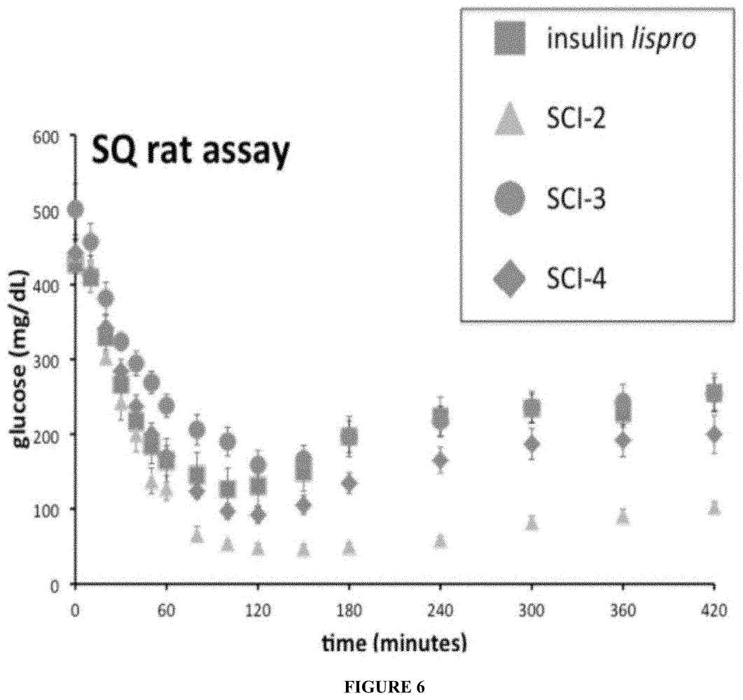

The pharmacodynamics features of this analogue were tested following subcutaneous injection in a rat model of diabetes mellitus as provided by Menting, J. G., et al. (2014, “Protective hinge in insulin opens to enable its receptor engagement.” Proc. Natl. Acad. Sci. USA 111(33):E3395-404). As shown in FIG. 6, the biological activity, onset of action, and duration of action of SCI-4 (SEQ ID NO: 4) were defined relative insulin lispro and two single-chain analogues containing the more complex C domain previously known in the art (Glu-Glu-Gly-Pro-Arg-Arg; EEGPRR; SEQ ID NO: 8). SCI-2 (SEQ ID NO: 5), which contained the same four substitutions in the A- and B domains as SCI-4, exhibited rapid onset of action (similar to insulin lispro) but a prolonged tail of insulin action. SCI-3 (SEQ ID NO: 6) was a variant of SCI-2 in which the wild-type residues were restored in the A domain (i.e., Thr^{A8} and Tyr^{A14}); this variant exhibited slower onset of action but its offset was similar to that of insulin lispro. Remarkably, the pharmacodynamic profile of SCI-4 is essentially identical to that of insulin lispro with respect to onset of action, offset of action, and integrated potency (area over the curve). The absence of a prolonged tail despite the presence of His^{A8} and Glu^{A14} demonstrates an interplay between C-domain sequence and modifications in the insulin moiety.

A method for treating a patient with diabetes mellitus comprises administering a single-chain insulin analogue as described herein. The insulin analogues of the present invention may be used as a medicament or for the treatment of disease. In some examples, the insulin analogues may be used in the manufacture of a medicament for the treatment of diabetes mellitus.

It is another aspect of the present invention that the single-chain insulin analogues may be prepared either in yeast (Pichia pastoris) or subject to total chemical synthesis by native fragment ligation. The synthetic route of preparation is preferred in the case of non-standard modifications, such as D-amino-acid substitutions, halogen substitutions within the aromatic rings of Phe or Tyr, or O-linked modifications of Serine or Threonine by carbohydrates; however, it would be feasible to manufacture a subset of the single-chain analogues containing non-standard modifications by means of extended genetic-code technology or four-base codon technology. It is yet another aspect of the present invention that use of non-standard amino-acid substitutions can augment the resistance of the single-chain insulin analogue to chemical degradation or to physical degradation. We further envision the analogues of the present invention providing a method for the treatment of diabetes mellitus or the metabolic syndrome. The route of delivery of the insulin analogue is typically by subcutaneous injection through the use of a syringe or pen device. An insulin pump may similarly be used, such as an external insulin pump or implantable intra-peritoneal pump.

A single-chain insulin analogue of the present invention may also contain other modifications, such as a halogen atom at positions B24, B25, or B26 as described more fully in U.S. Pat. No. 8,921,313, the disclosure of which is incorporated by reference herein. An insulin analogue of the present invention may also contain a foreshortened B-chain due to deletion of residues B1-B3.

A pharmaceutical composition may comprise such insulin analogues, and physiologically acceptable salts thereof, which may optionally include zinc. Zinc ions may be included at varying zinc ion:protein ratios, ranging from 2.2 zinc atoms per insulin analogue hexamer to 10 zinc atoms per insulin analogue hexamer. The pH of the formulation may either be in the range pH 3.0-4.5 (as a basal formulation of a pI-shifted single-chain insulin analogue) or be in the range pH 6.5-8.0 (as a prandial insulin formulation of a single-chain insulin analogue whose pI is similar to that of wild-type insulin). In either such formulation, the concentration of the insulin analogue would typically be between about 0.6-5.0 mM; concentrations up to 5 mM may be used in vial or pen; the more concentrated formulations (U-200 or higher, including in the range U-500 through U-1000) may be of particular benefit in patients with marked insulin resistance. Excipients may include glycerol, glycine, arginine, Tris, other buffers and salts, and anti-microbial preservatives such as phenol and meta-cresol; the latter preservatives are known to enhance the stability of the insulin hexamer. Such a pharmaceutical composition may be used to treat a patient having diabetes mellitus or other medical condition by administering a physiologically effective amount of the composition to the patient.

The amino-acid sequence of human proinsulin is provided, for comparative purposes, as SEQ ID NO: 1.

(human proinsulin)
SEQ ID NO: 1
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr-Arg-Arg-Glu-Ala-Glu-Asp-
Leu-Gln-Val-Gly-Gln-Val-Glu-Leu-Gly-Gly-Gly-Pro-
Gly-Ala-Gly-Ser-Leu-Gln-Pro-Leu-Ala-Leu-Glu-Gly-
Ser-Leu-Gln-Lys-Arg-Gly-Ile-Val-Glu-Gln-Cys-Cys-
Thr-Ser-Ile-Cys-Ser-Leu-Tyr-Gln-Leu-Glu-Asn-Tyr-
Cys-Asn
The amino-acid sequence of the A chain of human
insulin is provided as SEQ ID NO: 2.
(human A chain)
SEQ ID NO: 2
Gly-Ile-Val-Glu-Gln-Cys-Cys-Thr-Ser-Ile-Cys-Ser-
Leu-Tyr-Gln-Leu-Glu-Asn-Tyr-Cys-Asn
The amino-acid sequence of the B chain of human
insulin is provided as SEQ ID NO: 3.
(human B chain)
SEQ ID NO: 3
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr
The amino-acid sequence of SCI-4 is provided as
SEQ ID NO: 4.
SEQ ID NO: 4
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Asp-Pro-Thr-Glu-Ala-Ala-Ala-Ala-Ala-
Gly-Ile-Val-Glu-Gln-Cys-Cys-His-Ser-Ile-Cys-Ser-
Leu-Glu-Gln-Leu-Glu-Asn-Tyr-Cys-Asn
The amino-acid sequence of SCI-2 is provided as
SEQ ID NO: 5.
SEQ ID NO: 5
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Asp-Pro-Thr-Glu-Glu-Gly-Pro-Arg-Arg-
Gly-Ile-Val-Glu-Gln-Cys-Cys-His-Ser-Ile-Cys-Ser-
Leu-Glu-Gln-Leu-Glu-Asn-Tyr-Cys-Asn
The amino acid sequence of SCI-3 is provided as
SEQ ID NO: 6
SEQ ID NO: 6
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Asp-Pro-Thr-Glu-Glu-Gly-Pro-Arg-Arg-
Gly-Ile-Val-Glu-Gln-Cys-Cys-Thr-Ser-Ile-Cys-Ser-
Leu-Tyr-Gln-Leu-Glu-Asn-Tyr-Cys-Asn

Based upon the foregoing disclosure, it should now be apparent that an ultra-stable single-chain insulin analogue may be made compatible with unperturbed duration of insulin signaling through the co-engineering of a foreshortened and simplified C domain containing a poly-Alanine sub-segment. The resulting single-chain insulin analogues provided will carry out the objects set forth hereinabove.

What is claimed is:

1. A single-chain insulin analogue comprising the insulin A-chain polypeptide sequence, the insulin B-chain polypeptide sequence and a connecting peptide that connects the insulin A-chain polypeptide sequence to the B-chain polypeptide sequence, wherein the connecting peptide has the sequence of SEQ ID NO: 9.

2. The single-chain insulin analogue of claim 1, wherein the N-terminal amino acid of the connecting peptide is Glu.

3. The single-chain insulin analogue of claim 2, wherein the C-terminal amino acid of the connecting peptide is Ala.

4. The single-chain insulin analogue of claim 3, wherein the connecting peptide comprises five Ala residues.

5. The single-chain insulin analogue of claim 4, wherein the connecting peptide consists of Glu-Ala-Ala-Ala-Ala-Ala.

6. The single-chain insulin analogue of claim 1, wherein the C-terminal amino acid is Ala.

7. The single-chain insulin analogue of claim 6, wherein the connecting peptide comprises five Ala residues.

8. The single-chain insulin analogue of claim 7, wherein the connecting peptide consists of Xaa-Ala-Ala-Ala-Ala-Ala, wherein Xaa is Asp or Glu.

9. The insulin analogue of claim 8, wherein the insulin A-chain polypeptide sequence comprises a His substitution at the position corresponding to position A8 of human insulin, a Tyr substitution at the position corresponding to position A14 of human insulin, or both.

10. The insulin analogue of claim 9, wherein the B-chain polypeptide sequence comprises an Asp substitution at the position corresponding to position B28 of human insulin, a Pro substitution at the position corresponding to position B29 of human insulin, or both.

11. The insulin analogue of claim 8, wherein the B-chain polypeptide sequence comprises an Asp substitution at the position corresponding to position B28 of human insulin, a Pro substitution at the position corresponding to position B29 of human insulin, or both.

12. A method of lowering the blood sugar of a patient in need thereof, comprising administering a single-chain insulin analogue, or a physiologically acceptable salt thereof, to the patient, wherein the single chain insulin analogue comprises the insulin A-chain polypeptide sequence, the insulin B-chain polypeptide sequence and a connecting peptide that connects the insulin A-chain polypeptide sequence to the B-chain polypeptide sequence, wherein the connecting peptide has the sequence of SEQ ID NO: 9.

13. The method of claim 12, wherein the single-chain insulin analogue is administered by use of an insulin pen, an external insulin pump or implantable intra-peritoneal pump.

14. (canceled)

15. (canceled)

16. A nucleic acid encoding a single-chain insulin analogue comprising the insulin A-chain polypeptide sequence, the insulin B-chain polypeptide sequence and a connecting peptide that connects the insulin A-chain polypeptide sequence to the B-chain polypeptide sequence, wherein the connecting peptide has the of SEQ ID NO: 9.

17. The insulin analogue of claim 5, wherein the insulin A-chain polypeptide sequence comprises a His substitution at the position corresponding to position A8 of human insulin, a Tyr substitution at the position corresponding to position A14 of human insulin, or both.

18. The insulin analogue of claim 17, wherein the B-chain polypeptide sequence comprises an Asp substitution at the position corresponding to position B28 of human insulin, a Pro substitution at the position corresponding to position B29 of human insulin, or both.

19. The insulin analogue of claim 3, wherein the insulin A-chain polypeptide sequence comprises a His substitution at the position corresponding to position A8 of human insulin, a Tyr substitution at the position corresponding to position A14 of human insulin, or both.

20. The insulin analogue of claim 19, wherein the B-chain polypeptide sequence comprises an Asp substitution at the position corresponding to position B28 of human insulin, a Pro substitution at the position corresponding to position B29 of human insulin, or both.

21. The method of claim 12, wherein the N-terminal amino acid of the connecting peptide is Glu.

22. The method of claim 21, wherein the C-terminal amino acid of the connecting peptide is Ala.


SEQUENCE LISTING (13 sequences)

SEQ ID NO: 1 - protein, 86 aa, Homo sapiens
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 2 - protein, 21 aa, Homo sapiens
GIVEQCCTSICSLYQLENYCN

SEQ ID NO: 3 - protein, 30 aa, Homo sapiens
FVNQHLCGSHLVEALYLVCGERGFFYTPKT

SEQ ID NO: 4 - protein, 57 aa, Artificial Sequence
  SYNTHETIC CONSTRUCT
FVNQHLCGSHLVEALYLVCGERGFFYTDPTEAAAAAGIVEQCCHSICSLEQLENYCN

SEQ ID NO: 5 - protein, 57 aa, Artificial Sequence
  SYNTHETIC CONSTRUCT
FVNQHLCGSHLVEALYLVCGERGFFYTDPTEEGPRRGIVEQCCHSICSLEQLENYCN

SEQ ID NO: 6 - protein, 57 aa, Artificial Sequence
  SYNTHETIC CONSTRUCT
FVNQHLCGSHLVEALYLVCGERGFFYTDPTEEGPRRGIVEQCCTSICSLYQLENYCN

SEQ ID NO: 7 - protein, 6 aa, Artificial Sequence
  SYNTHETIC CONSTRUCT
GGGPRR

SEQ ID NO: 8 - protein, 6 aa, Artificial Sequence
  SYNTHETIC CONSTRUCT
EEGPRR

SEQ ID NO: 9 - protein, 11 aa, Artificial Sequence
  SYNTHETIC CONSTRUCT
  MISC_FEATURE (1)..(1): X is Asp or Glu
  MISC_FEATURE (3)..(9): X is 0-7 Ala residues
  MISC_FEATURE (11)..(11): X is Ala, Arg, Asn, Asp, Glu, His, Lys, or Ser
XAXXXXXXXAX

SEQ ID NO: 10 - protein, 30 aa, Artificial Sequence
  SYNTHETIC CONSTRUCT
FVNCHLCGSDLVEALYLVCGERGFFYTKPT

SEQ ID NO: 11 - protein, 21 aa, Artificial Sequence
  SYNTHETIC CONSTRUCT
GIVEQCCTSCCSLYQLENYCN

SEQ ID NO: 12 - protein, 30 aa, Artificial Sequence
  SYNTHETIC CONSTRUCT
FVNQHLCGSHLVEALYLVCGERGFFYTKPT

SEQ ID NO: 13 - protein, 30 aa, Artificial Sequence
  SYNTHETIC CONSTRUCT
FVNQHLCGSDLVEALYLVCGERGFFYTKPT